(19) United States
(12) Patent Application Publication
(10) Pub. No.: US 2018/0031483 A1
(43) Pub. Date: Feb. 1, 2018

(54) BIOPLASMONIC DETECTION OF BIOMARKERS IN BODY FLUIDS USING PEPTIDE RECOGNITION ELEMENTS

(71) Applicant: Washington University in St. Louis, St. Louis, MO (US)

(72) Inventors: Srikanth Singamaneni, St. Louis, MO (US); Sirimuvva Tadepalli, St. Louis, MO (US); Jeremiah J. Morrissey, St. Louis, MO (US); Evan D. Kharasch, University City, MO (US)

(21) Appl. No.: 15/531,205

(22) PCT Filed: Nov. 24, 2015
(86) PCT No.: PCT/US2015/062285

§ 371 Date: May 26, 2017

Related U.S. Application Data
(60) Provisional application No. 62/084,763, filed on Nov. 26, 2014.

(51) Int. Cl. G01N 21/65; G01N 33/543; G01N 33/68; G01N 21/552

(52) U.S. Cl. CPC G01N 21/658; G01N 21/554; G01N 33/54373; G01N 33/6893; G01N 33/54346; G01N 2333/4712

(57) ABSTRACT

Plasmonic nanotransducers and methods for label-free detection of biomarkers are disclosed. The plasmonic nanotransducers include nanostructure cores and peptide aptamers. The plasmonic nanotransducers are exposed to a biological sample that can contain the specific biomarkers and can be analyzed with surface enhanced Raman scattering techniques and/or localized surface plasmon resonance techniques to quantify the amount of the biomarker in the sample.

### CROSS-REFERENCE TO RELATED APPLICATIONS

This application claims priority to U.S. Provisional Patent Application No. 62/084,763, filed on Nov. 26, 2014, which is hereby incorporated by reference in its entirety.

### GOVERNMENT RIGHTS IN THE INVENTION

This invention was made with government support under grant number NCIR01CA141521 awarded by the National Institutes of Health and CBET-1254399 awarded by the National Science Foundation. The government has certain rights in the invention.

### FIELD OF THE INVENTION

The present disclosure relates generally to compositions and methods for detecting biomarkers in fluidic samples. More particularly, the present disclosure relates to peptide-based plasmonic biosensors and label-free methods to detect biomarkers in fluidic samples using the peptide-based plasmonic biosensors.

### BACKGROUND

Biosensing platforms based on localized surface plasmon resonance (LSPR) or surface enhanced Raman scattering (SERS) hold enormous potential to provide highly sensitive, cost-effective, and point-of-care diagnostic tools. However, similar to many other analytical methodologies such as enzyme-linked immunosorbent assays (ELISAs), present plasmonic biosensors use natural antibodies. The use of natural antibodies in analytical methods is ubiquitous, with applications in disease diagnosis, toxicology testing, and biotechnology. Natural antibody production is expensive and time consuming. Both the time and expense required for natural antibody production and their poor stability constitute a barrier to the rapid development and widespread application of plasmonic biosensors and clinical protocols for disease-specific screening.

Although gold nanoparticles may enable LSPR spectroscopy and improve sensitivity, they have so far been used as a layer underneath or on top of a molecularly imprinted polymer (MIP) film. In these configurations, nanoparticles are not used as direct transduction elements but for enhancing Raman scattering from analyte molecules (SERS) or propagating surface plasmon resonance (SPR) on planar gold surfaces. Other reported techniques involve embedding gold nanoparticles in a molecularly imprinted polymer or so-called Au-MIP nanocomposites, which results in a random distribution of the nanoparticles and the molecular imprints. Biomacromolecular imprinting of noble-metal nanoparticles that takes full advantage of the unique structural and localized plasmonic properties of each individual nanoparticle continues to be a serious challenge. Present metal nanostructures have low refractive index sensitivity, which can impede detection of biomolecules at low concentrations.

Most of the existing plasmonic sensors rely on natural antibodies for the capture of target biomolecules (e.g., disease biomarkers). However, natural antibodies suffer from numerous shortcomings such as poor chemical stability, excessive cost and limited shelf-life. Moreover, they pose a significant challenge in efficient integration with abiotic microtransduction and nanotransduction platforms.

Accordingly, there is a need for sensors with a higher refractive index sensitivity for plasmonic biosensing. In addition, there is a need for alternative methods for detecting biomarkers.

### BRIEF DESCRIPTION OF THE DISCLOSURE

The present disclosure relates generally to compositions and methods for detecting biomarkers in fluidic samples. More particularly, the present disclosure relates to peptide-based plasmonic biosensors and label-free methods to detect biomarkers in fluidic samples using the peptide-based plasmonic biosensors.

In one aspect, the present disclosure is directed to a plasmonic nanotransducer comprising a nanostructure core and at least one peptide coupled to the nanostructure core, wherein the at least one peptide specifically binds to a target molecule.

In one aspect, the present disclosure is directed to a plasmonic nanotransducer comprising a hollow nanostructure core and at least one peptide coupled to the hollow nanostructure core, wherein the at least one peptide specifically binds to a target molecule.

In another aspect, the present disclosure is directed to a label-free method for detecting a target molecule in a biological sample. The method comprises obtaining a biological sample from the subject; contacting the biological sample with a plasmonic nanotransducer, wherein the plasmonic nanotransducer comprises: a nanostructure core and at least one peptide coupled to the nanostructure core, wherein the at least one peptide specifically binds to a target molecule; wherein the target molecule in the biological sample forms a complex with the plasmonic nanotransducer; and detecting the complex.

In another aspect, the present disclosure is directed to a label-free method for detecting a target molecule in a biological sample. The method comprises obtaining a biological sample from the subject; contacting the biological sample with a plasmonic nanotransducer, wherein the plasmonic nanotransducer comprises: a hollow nanostructure core and at least one peptide coupled to the hollow nanostructure core, wherein the at least one peptide specifically binds to a target molecule; wherein the target molecule in the biological sample forms a complex with the plasmonic nanotransducer; and detecting the complex.

### BRIEF DESCRIPTION OF THE DRAWINGS

The following figures illustrate various aspects of the disclosure.

FIG. 1A is a schematic showing the effect of the distance of a peptide recognition element from the surface of the plasmonic nanotransducer on the refractive index sensitivity.

FIG. 1B is a schematic showing the effect of the distance of an antibody recognition element from the surface of the plasmonic nanotransducer on the refractive index sensitivity.

FIG. 2 is a transmission electron micrograph image of AuNRs used as nanotransducers. Scale bar represents 50 nm.

FIG. 3 depicts the extinction spectra showing the LSPR shift after conjugation of AuNR with troponin binding peptide. Inset shows the magnified image of the shift.

FIG. 4 depicts the AFM height profile of NR, NR associated with peptide, and NR with peptide and troponin binding.

FIG. 5 is an image showing paper without peptide aptamer-based AuNR (left) and paper with peptide aptamer-based AuNR (right).

FIG. 6 is a SEM image of peptide aptamer-based AuNR showing uniform distribution of the AuNR. Scale bar represents 1 μm.

FIG. 7 depicts extinction spectra of peptide aptamer-based AuNR after absorption on paper at different locations showing a homogenous LSPR with a standard deviation of less than 1 nm.

FIG. 8 depicts a representative LSPR spectrum from the paper substrate deconvoluted using two peak Gaussian fit.

FIG. 9 depicts the LSPR Spectra showing a blue shift in peptide conjugated NR (AuNR+Peptide) in solution to peptide conjugated NR (AuNR+Peptide) absorbed on paper because of the refractive index change.

FIG. 10 depicts the Raman spectrum taken from paper substrates confirming the peptide conjugation on the AuNR surface as compared to AuNR alone.

FIG. 11 depicts a dot-blot using pegylated and unpegylated anti-Troponin (cTn1) antibodies to confirm that the affinity towards recombinant human cTn1 (increasing concentrations from left to right) was not altered during the process of conjugation with EDC-NHS to give a thiol terminus to the antibody.

FIG. 12 depicts the hydrodynamic radius of AuNRs after peptide aptamer (AuNR+Peptide) and antibody (AuNR+Antibody) coupling.

FIG. 13 depicts AFM images of AuNR, AuNR+Peptide aptamer, and AuNR+Antibody.

FIG. 14 depicts the height profile of the AuNR following successful coupling showing the difference in the height of the peptide aptamer recognition element (NR+Peptide) and the antibody recognition element (NR+Antibody).

FIG. 15 depicts extinction spectra showing LSPR shift after Troponin binding with antibody conjugated AuNR (AuNR+H-antibody+3.53 μg/ml Troponin) as compared to AuNR+H-antibody alone.

FIG. 16 depicts extinction spectra showing the LSPR shift after Troponin binding with peptide aptamer conjugated AuNR (AuNR+Peptide+3.53 μg/ml Troponin) as compared to AuNR+Peptide alone.

FIG. 17 is a schematic showing layer-by-layer adsorption of PSS and PAH on the surface of AuNR on a glass substrate.

FIG. 18 depicts the distance dependent refractive index sensitivity of AuNRs adsorbed on a glass substrate.

FIG. 19 depicts Troponin sensing of antibody conjugated AuNR and peptide conjugated AuNR at different troponin concentrations (pg/ml).

FIG. 20 depicts the distance dependent sensitivity (σ) of AuNRs adsorbed on a glass substrate showing different sensitivity (σ) for antibody conjugated AuNR and peptide conjugated AuNR. The σ values obtained from the curve for peptide conjugated AuNR is 6.67 nm/nm and antibody conjugated AuNR is 4.33 nm/nm. The corresponding shift for peptide conjugated AuNR from this curve is 24.5 nm whereas from antibody conjugated AuNR is 18 nm. The ratio of the shifts corresponding to peptide conjugated AuNR and antibody conjugated AuNR is 1.4. The ratio of the shifts from peptide conjugated AuNR to antibody conjugated AuNR upon exposure to 3.53 μg/ml troponin is 1.9.

FIG. 21 depicts LSPR shift at different concentrations of Troponin and Human Serum Albumin showing that the selectivity of antibody conjugated AuNR is better than that of the peptide conjugated AuNR.

FIG. 22 depicts the LSPR shift for antibody conjugated AuNR and peptide conjugated AuNR after exposure to the 3.53 μg/ml Troponin sample after 48 hour treatment at 4° C. and 60° C.

FIG. 23 depicts the sensing calibration curve of Troponin spiked in human serum ( 1/10th concentration) in buffer.

FIG. 24 depicts the sensing calibration curve of Troponin spiked in artificial sweat ( 1/10th concentration) in buffer.

### DETAILED DESCRIPTION

Provided herein are plasmonic nanotransducers and methods for label-free plasmonic biosensing using plasmonic nanotransducers. The refractive index sensitivity of localized surface plasmon resonance (LSPR) of plasmonic nanostructures renders them an attractive transduction platform for chemical and biological sensing to detect biomarkers for a wide variety of diseases.

### Definitions

Provided herein are plasmonic nanotransducers and methods for label-free plasmonic biosensing using plasmonic nanotransducers. The refractive index sensitivity of localized surface plasmon resonance (LSPR) of plasmonic nanostructures renders them an attractive transduction platform for chemical and biological sensing to detect biomarkers for a wide variety of diseases.

As used herein, the term “targeted polypeptide” refers to “native sequence” polypeptides and variants (which are further defined herein).

A “native sequence” polypeptide includes a polypeptide having the same amino acid sequence as the corresponding polypeptide derived from nature. Thus, the term “native sequence polypeptide” includes naturally-occurring truncated, augmented, and frame-shifted forms of a polypeptide, including alternatively spliced forms, isoforms and polymorphisms.

As used herein, the term “naturally occurring variant” refers to a polypeptide having at least about 60% amino acid sequence identity with a reference polypeptide and retains at least one biological activity of the naturally occurring reference polypeptide. Naturally occurring variants can include variant polypeptides having about 65% amino acid sequence identity, about 70% amino acid sequence identity, about 75% amino acid sequence identity, about 80% amino acid sequence identity, about 80% amino acid sequence identity, about 85% amino acid sequence identity, about 90% amino acid sequence identity, about 95% amino acid sequence identity, about 98% amino acid sequence identity or about 99% amino acid sequence identity to a reference polypeptide.

As used herein, the term “detection” includes any methods of detecting, including direct and indirect detection.

The term “molecular subtype,” is used interchangeably herein with “molecular phenotype,” to refer to a subtype or phenotype of a disease or condition characterized by the expression of one or more particular genes or one or more particular proteins, or a particular pattern of expression of a combination of genes or a combination of proteins. The expression of particular genes, proteins or combinations of genes or proteins can be further associated with certain pathological, histological, and/or clinical features of the disease or condition.

As used herein, the term “biomarker” refers to an indicator of, for example, a pathological state of a subject, which can be detected in a biological sample of the subject. Biomarkers include DNA-based, RNA-based and protein-based molecular markers.

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition (such as idiopathic pulmonary fibrosis) or be diagnosed with a condition (such as idiopathic pulmonary fibrosis). The subject can also be suspected of having or at risk for having a lung disease or be diagnosed with a lung disease such as, for example, hypersensitivity pneumonitis, cryptogenic cryptogenic organizing pneumonia, diffuse alveolar damage, chronic obstructive pulmonary disease, chronic bronchitis, pulmonary emphysema, pulmonary arterial hypertension, nonspecific interstitial pneumonitis, systemic sclerosis associated interstitial lung disease, or collagen vascular disease-associated interstitial lung disease. According to one embodiment, the subject to be treated according to this invention is a human.

### I. Plasmonic Nanotransducers for Plasmonic Biosensing

In one aspect, the present disclosure is directed to a plasmonic nanotransducer. The plasmonic biosensor includes a nanostructure core and at least one aptamer coupled to the nanostructure core, wherein the at least one aptamer specifically binds to at least one biomarker.

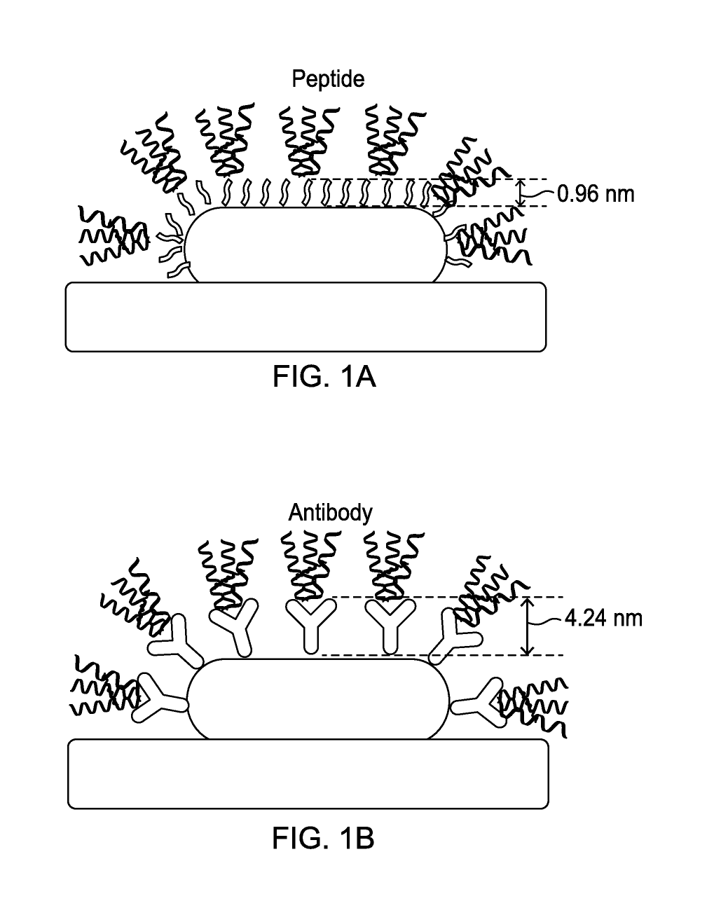

The response of an LSPR nanotransducer can be described by the equation (1):

$$R = m Δ η ( 1 - {e}^{\frac{- d}{{l}_{d}}} ) \qquad ( 1 )$$

where R is the LSPR shift, m is the refractive index sensitivity (RIS), Δη is the difference in the refractive index between the adsorbed layer and the medium, d is the layer thickness and l_{d} is the plasmon decay length. Thus the LSPR response of a biosensor depends upon the RIS and the decay length which are characteristic to a given nanotransducer. However, in the presence of a recognition element which is adsorbed on the nanotransducer, the LSPR response is given by equation (2):

$$R = m Δ η {e}^{\frac{- {d}_{1}}{{l}_{d}}} ( 1 - {e}^{\frac{- {d}_{2}}{{l}_{d}}} ) \qquad ( 2 )$$

where d₁ and d₂ are the thicknesses of the recognition element and the analyte layer respectively. The EM decay length (l_{d}) should be chosen to encompass d₁ and d₂. For a nanoparticle of a characteristic decay length that encompasses the recognition element and the analyte layer, as shown in the schematics in FIGS. 1A and 1B, the LSPR shift is higher in the case of a smaller recognition element according to equation (2).

Suitable nanostructure cores can be selected from nanoparticles, nanorods, nanotubes, nanobipyramids, nanocubes, nanocages, nanostars, nano-octahedra, nanoshells, nanorattles, nanomatryoshkas and any other nanostructure to which the at least one peptide can be coupled. Particularly suitable nanostructure cores can be hollow nanostructure cores such as, for example, nanocages, nanorattles, nanoshells, nanomatryoshkas and combinations thereof. Nanostructure cores can be, for example, gold nanostructure cores, silver nanostructure cores, copper nanostructure cores, and combinations thereof.

Aptamer that specifically bind to at least one biomarker can be a peptide aptamer. Peptide aptamer selection can be made using different systems such as, for example, yeast two-hybrid system and combinatorial peptide libraries constructed by phage display, mRNA display, ribosome display, bacterial display and yeast display.

Phage display is a particularly suitable method for preparing the peptide aptamers. In phage display, a gene encoding a target protein of interest is inserted into a phage coat protein gene causing the bacteriophage to display the protein on its outside. These displaying phages can then be screened against aptamers to detect interactions between the displayed target protein and the aptamers. By immobilizing a target molecule to the surface of a microtiter plate well, a phage that displays a protein that binds to one of those targets on its surface will remain while others can be removed by washing. Phage that remain can be eluted and used to produce more phage to produce a phage mixture that is enriched with relevant (i.e. binding) phage. The repeated cycling of these steps (panning) results in the enrichment of aptamers with higher affinity for the target molecule (e.g., biomarkers). Phage eluted in the final step can be used to infect a suitable bacterial host, from which the phagemids can be collected and the relevant DNA sequence excised and sequenced to identify the relevant, interacting proteins or protein fragments. Sequences of the aptamer can be obtained according to methods known to those skilled in the art. The aptamer can then be prepared using methods known to those skilled in the art such as chemical synthesis and recombinant protein expression.

Peptide aptamer can further include a linker amino acid sequence to link the peptide aptamer to the nanostructure. Any desired length of linker can be added to the peptide aptamer to function as a spacer between the nanostructure surface and the peptide aptamer such that the peptide aptamer can interact with the biomarker. The linker can be added to the peptide aptamer sequence at the N-terminus and/or at the C-terminus of the peptide aptamer sequence. Suitable linkers can be, for example, a single cysteine amino acid residue attached to a series of consecutive glycine amino acid residues. Suitable linkers can also include more than one consecutive cysteine residues attached to one to five consecutive glycine residues. A particularly suitable linker can include a single cysteine amino acid residue attached to a series of three consecutive glycine amino acid residues. The cysteine residue of the linker binds to the nanostructure and the last glycine residue is coupled to the N-terminus or C-terminus of the peptide aptamer. Preferably, the linker is coupled to the C-terminus of the peptide aptamer.

The plasmonic biosensors can further be adsorbed to a substrate. Suitable substrates can be, for example, glass substrates, paper substrates, and fibrous mats. Particularly suitable glass substrates can be, for example, silica, titania, alumina and combinations thereof. Particularly suitable paper substrates can be, for example, cellulose paper, nitrocellulose paper, methylcellulose paper, hydroxypropylcellulose paper, and nanocellulose paper. Particularly suitable fibrous mats can be, for example, woven fibrous mats and non-woven fibrous mats.

The biomarkers can be any biomarker that can be measured and evaluated to examine normal biological processes, disease, pathogenic processes, and pharmacologic responses to a therapeutic intervention. Disease-related biomarkers can be, for example, predictive biomarkers (risk indicators), diagnostic biomarkers, and prognostic biomarkers. Predictive biomarkers can provide an indication of the probable effect of treating a patient. Diagnostic biomarkers can provide an indication whether a disease already exists. Prognostic biomarkers can provide an indication of how a disease may develop in an individual case regardless of the type of treatment. Predictive biomarkers help to assess the most likely response to a particular treatment type, while prognostic markers show the progression of disease with or without treatment. Suitable biomarkers also include drug-related biomarkers that indicate whether a drug will be effective in a specific patient and how the patient will process the drug. Suitable biomarkers include, for example, cardiac biomarkers, cancer biomarkers, kidney disease biomarkers, aging biomarkers, hospital-acquired infection biomarkers, and food poisoning biomarkers.

Suitable biomarkers can be biomarkers known for diagnosing acute myocardial infarction (MI). Particularly suitable biomarkers for MI include, for example, troponins such as troponin I (cTI), fatty acid-binding protein 3 (FABP3; also known as Heart-type fatty acid-binding protein (H-FABP)), creatine kinase-MB, lactate dehydrogenase, aspartate transaminase, myoglobin, ischemia-modified albumin, B-type natriuretic peptide (BNP), N-terminal fragment of pro-BNP (NT-proBNP), Mid-regional pro-Atrial Natriuretic Peptide, glycogen phosphorylase isoenzyme BB, soluble urokinase-type plasminogen activator receptor, copeptin, myeloperoxidase (MPO), growth differentiation factor 15 (GDF-15), high sensitivity C-reactive protein (hsCRP), placental growth factor (P1GF), whole blood choline (WBCIIO), interleukin 1 receptor-like 1 (ST2), C-Terminal pro-endothelin 1, Mid-regional pro-Adrenomedullin, and combinations thereof.

Suitable biomarkers can be biomarkers known for diagnosing kidney injury and kidney disease. Particularly suitable biomarkers for kidney injury and kidney disease include, for example, serum creatinine (SCr), cystatin C (CyC), neutrophil gelatinase-associated lipocalin (NGAL), kidney injury molecule-1 (KIM-1), β-Trace protein (BTP), uric acid (UA), proteinuria, albumin, liver-fatty acid binding protein (L-FABP), interleukin-18 (IL-18), urine cystatin C (uCyC), Alpha-glutathione s-transferase (α-GST), pi-glutathione s-transferase (π-GST), gammaglutanyl transpeptidase (GGT), alkaline phosphatase (AP), N-acetyl-β-D-glucosaminidase (NAG), tenascin, tissue inhibitor of metalloproteinases 1, nephrin, podocin, podocalyxin, asymmetric dimethylarginine (ADMA), C-reactive protein (CRP), soluble tumor nectosis factor receptor II, pentraxin-3 (PTX3), transforming growth factor-β1 (TGF-β1), CD14, fibroblast growth factor-23 (FGF-23), apolipoprotein A-IV, adiponectin, γ-glutamyl transpeptidase (GGT), Tumor necrosis factor-related apoptosis-inducing ligand (TRAIL), bone morphogenetic protein-7 (BMP-7), and combinations thereof.

Suitable biomarkers can be biomarkers known for diagnosing cancer. Particularly suitable biomarkers for cancer include, for example, alpha-fetoprotein (AFP) for Liver Cancer, breakpoint cluster region-Abelson murine leukemia viral oncogene homolog 1 (BCR-ABL) for Chronic Myeloid Leukemia, breast cancer 1 (BRCA1)/breast cancer 2 (BRCA2) for Breast/Ovarian Cancer, V-Raf Murine Sarcoma Viral Oncogene Homolog B1 (BRAF V600E) for Melanoma and Colorectal Cancer, cancer antigen-125 (CA-125) for Ovarian Cancer, carbohydrate antigen 19-9 (CA19.9) for Pancreatic Cancer, carcinoembryonic antigen (CEA) for Colorectal Cancer, epidermal growth factor receptor (EGFR) for Non-small-cell lung carcinoma, human epidermal growth factor receptor 2 (HER-2) for Breast Cancer, Mast/stem cell growth factor receptor (KIT or CD117) for Gastrointestinal stromal tumor, prostate-specific antigen (PSA) for Prostate Cancer, S100 for Melanoma, fatty acid-binding protein 3 (FABP3) for brain tumors, aquaporin-1 (AQP1) and perilipin 2 (PLIN2) for kidney cancer, and combinations thereof.

Suitable biomarkers can be biomarkers known for diagnosing hospital-acquired infections. Particularly suitable biomarkers for hospital-acquired infections include, for example, biomarkers for Staphylococcus aureus infections such as, for example, community-associated (CA) methicillin-resistant Staphylococcus aureus (MRSA), hospital-associated (HA) MRSA, and vancomycin-intermediate S. aureus (VISA). Suitable biomarkers for Staphylococcus aureus infections include, for example, enterotoxins (see Toxicon 2002; 40:1723-1726), serum C-reactive protein, anti-glucosaminidase IgG, alpha toxin, acyl carrier protein, phenol-soluble modulin α1 and phenol-soluble modulin α2 peptides, and combinations thereof.

Suitable biomarkers can be biomarkers known for detecting Escherichia coli O157:H7 food poisoning, which can lead to hemolytic uremic syndrome.

Individual plasmonic biosensors can be prepared with at least one peptide that specifically binds to a biomarker of interest. Individual plasmonic biosensors specific for a biomarker of interest can be combined with other plasmonic biosensors prepared with at least one peptide that specifically binds to a different biomarker of interest to obtain a combination of plasmonic biosensors for detecting multiple biomarkers.

II. Plasmonic Nanotransducers having Hollow Nanostructure Cores for Plasmonic Biosensing

In one aspect, the present disclosure is directed to a plasmonic nanotransducer. The plasmonic biosensor includes a nanostructure core and at least one aptamer coupled to the nanostructure core, wherein the at least one aptamer specifically binds to at least one biomarker.

Suitable hollow nanostructure cores such as, for example, nanocages, nanorattles, nanoshells, nanomatryoshkas and combinations thereof. Non-limiting examples of nanostructures for preparing hollow nanostructure cores include, for example, nanoparticles, nanocages, nanorods, nanobipyramids, nanostars, nano-octahedra, nanorattles and any other nanostructure. In an aspect, the hollow nanostructure core of the plasmonic nanotransducer can be a gold nanocage. In an aspect, the hollow nanostructure core of the plasmonic nanotransducer can be a gold nanorattle. In another aspect, the hollow nanostructure core may be a gold nanorod.

The hollow nanostructure core of the plasmonic nanotransducer includes a metal nanostructure core further including a porous metal shell. Hollow nanostructure cores can be prepared by coating a nanostructure core (e.g., nanoparticles, nanocubes, nanorods, nanobipyramids, nanostars, and nano-octahedra) with a metal to form a metal shell surrounding the nanostructure core. The metal shell can then be treated to form pores in the metal shell, and result in the formation of the hollow nanotransducer. In an exemplary aspect, a gold nanostructure core such as nano-octahedra can be coated with silver to form a bi-metallic core-shell nanostructure having a silver metal shell on the gold nanostructure core. The silver metal shell can then be treated such as using galvanic replacement reaction to convert the silver metal shell into a porous gold shell. The nanostructure core remains embedded in the porous shell. Average pore size in the shell can be about 3 nm. Pore sizes can be determined using transmission electron microscopy.

Nanocages can be about 60 nm per side. AuNCs (gold nanocages) have a highly tunable LSPR into the near infrared (NIR), where the endogenous absorption coefficient of living tissue can be nearly two orders magnitude smaller compared to that in the visible range. Plasmonic biosensors including hollow nanostructure cores can exhibit higher refractive index sensitivity and lower electromagnetic (EM) decay length. Without being limited to a particular theory, the higher refractive index enables lowering the limit of detection (LOD) of the target biomarkers.

The plasmonic biosensors including hollow nanostructure cores and peptide aptamers can further be adsorbed to a substrate. Suitable substrates can be, for example, glass substrates, paper substrates, and fibrous mats. Particularly suitable glass substrates can be, for example, silica, titania, alumina and combinations thereof. Particularly suitable paper substrates can be, for example, cellulose paper, nitrocellulose paper, methylcellulose paper, hydroxypropylcellulose paper, and nanocellulose paper. Particularly suitable fibrous mats can be, for example, woven fibrous mats and non-woven fibrous mats.

Suitable biomarkers are described herein.

Plasmonic biosensors including hollow nanostructure cores and peptide aptamers can further include a linker as described herein.

### III. Label-Free Methods for Plasmonic Biosensing

In another aspect, the present disclosure is directed to a label-free method for detecting a biomarker in a biological sample. The method comprises obtaining a biological sample from the subject; contacting the biological sample with a plasmonic nanotransducer, wherein the plasmonic nanotransducer comprises: a nanostructure core and at least one peptide aptamer coupled to the nanostructure core, wherein the at least one peptide aptamer specifically binds to a biomarker; wherein the biomarker in the biological sample forms a complex with the plasmonic nanotransducer; and detecting the complex.

The nanostructure core of the plasmonic transducers can be any of those described herein. Suitable nanostructure cores can be selected from nanoparticles, nanorods, nanotubes, nanobipyramids, nanocubes, nanocages, nanostars, nano-octahedra, nanoshells, nanorattles, nanomatryoshkas and any other nanostructure to which the at least one peptide can be coupled as described herein. Particularly suitable nanostructure cores can be hollow nanostructure cores such as, for example, nanocages, nanorattles, nanoshells, nanomatryoshkas and combinations thereof as described herein. Nanostructure cores can be, for example, gold nanostructure cores, silver nanostructure cores, copper nanostructure cores, and combinations thereof as described herein.

In another aspect, the present disclosure is directed to a label-free method for detecting a biomarker in a biological sample. The method comprises obtaining a biological sample from the subject; contacting the biological sample with a plasmonic nanotransducer, wherein the plasmonic nanotransducer comprises: a hollow nanostructure core and at least one peptide aptamer coupled to the hollow nanostructure core, wherein the at least one peptide aptamer specifically binds to a biomarker; wherein the biomarker in the biological sample forms a complex with the plasmonic nanotransducer; and detecting the complex.

The nanostructure core of the plasmonic transducers can be any of those described herein. Suitable nanostructure cores can be selected from nanoparticles, nanorods, nanotubes, nanobipyramids, nanocubes, nanocages, nanostars, nano-octahedra, nanoshells, nanorattles, nanomatryoshkas and any other nanostructure to which the at least one peptide can be coupled as described herein. Particularly suitable nanostructure cores can be hollow nanostructure cores such as, for example, nanocages, nanorattles, nanoshells, nanomatryoshkas and combinations thereof as described herein. Nanostructure cores can be, for example, gold nanostructure cores, silver nanostructure cores, copper nanostructure cores, and combinations thereof as described herein.

The methods can further include detecting the complex using local surface plasmon resonance (LSPR) and surface enhanced Raman scattering (SERS). Surface plasmon involves the collective coherent oscillation of the conductive electrons at the interface of metal and dielectric materials.

Detecting the LSPR wavelength can include directing a light onto the plasmonic nanotransducers and detecting the reflected light using a spectrophotometer. In an aspect, the light can be ambient light. A phase shift in the LSPR wavelength from before contact with the sample and after contact with the sample can indicate binding of the target molecule to the plasmonic nanotransducer.

SERS spectra can be collected using methods known to those skilled in the art. For example, a confocal Raman spectrometer mounted on a Leica microscope equipped with 514.5 and 785 nm lasers and a hand-held spectrometer can be used. For a 785 nm wavelength laser, the focal volume (and spot diameter) of the laser focused with 20× and 50× objectives is 32.3 fl (1.20 μm) and 2.61 fl (0.64 μm), respectively. For moderate detection levels (concentration>1 μg/ml), SERS provides distinct spectral differences due to the strong Raman bands, which are enhanced 10⁵-10⁹ times compared to normal Raman scattering. To achieve the trace level analysis (concentration<100 ng/ml), multivariable statistical means, such as principal component analysis (PCA) via intrinsic Raman spectra of the analyte of interest, can be employed. Specifically, linear multivariable models of SERS spectra data sets can be built by establishing principal component vectors (PCs), which can provide the statistically most significant variations in the data sets, and reduce the dimensionality of the sample matrix. This approach involves assigning a score for the PCs of each spectrum collected followed by plotting the spectrum as a single data point in a two-dimensional plot. The plot reveals clusters of similar spectra, thus individual biological species (analyte and interfering molecules) can be classified and differentiated.

The biological sample can be a liquid biological sample. Suitable liquid biological samples can be, for example, whole blood, plasma, serum, urine, saliva, cerebrospinal fluid, and sweat. In an aspect, the liquid biological sample can be a cell extract such as a cell homogenate.

Suitable biomarker can be those biomarkers described herein.

### EXAMPLES

The following examples are included to demonstrate preferred embodiments of the invention. It should be appreciated by those of skill in the art that the techniques disclosed in the examples that follow represent techniques discovered by the inventors to function in the practice of the invention, and thus can be considered to constitute preferred modes for its practice. However, those of skill in the art should, in light of the present disclosure, appreciate that changes can be made in the specific embodiments which are disclosed and obtain a like or similar result without departing from the scope of the invention.

### Materials

Cetyltrimethylammonium bromide (CTAB), chloroauric acid, ascorbic acid, sodium borohydride, tris(hydroxymethyl)amino methane (tris), Albumin from Human Serum, poly(stryrene sulfonate) (PSS) (Mw=70,000 g/mol) and poly(allyl amine hydrochloride) (PAH) (Mw=56,000 g/mol) were purchased from Sigma Aldrich. Silver nitrate and filter paper (Whatman #1) was purchased from VWR international. 1-Ethyl-3-(3-dimethylaminopropyl) carbodiimide (EDC) and N-hydroxy succinimide (NHS) were purchased from Thermo Scientific. SH-PEG-COOH (Mw=5000 g/mole) was purchased from Jenkem Technology. Troponin I-C (H-41) was purchased from SantaCruz Biotechnology. Concentrated phosphate buffer saline (PBS) 10× was purchased from Omnipur. The Peptide Sequence was ordered from Genscript USA Inc., Human Cardiac Troponin-I Recombinant was purchased from Life Diagnostics, USA. Artificial eccrine was purchased from Pickering Laboratorie. All chemicals were used as received with no further purification.

Synthesis of gold nanorods (AuNRs).

Gold nanorods were synthesized using seed-mediated approach. Seed solution was prepared by adding 0.6 ml of an ice-cold sodium borohydride solution (10 mM) into 10 ml of 0.1 M cetyltrimethylammonium bromide (CTAB) and 2.5×10⁻⁴ M chloroauric acid (IIAuCl₄) solution under vigorous stirring at 25° C. The color of the seed solution changed from yellow to brown. Growth solution was prepared by mixing 95 ml of CTAB (0.1 M), 0.5 ml of silver nitrate (10 mM), 4.5 ml of HAuCl₄ (10 mM), and 0.55 ml ascorbic acid (0.1 M) consecutively. The solution was homogenized by gentle stirring. To the resulting colorless solution, 0.12 ml of freshly prepared seed solution was added and set aside in the dark overnight. Prior to use, the AuNRs solution was resuspended in nanopure water (18.2 MΩ·cm).

AuNR-Peptide aptamer conjugation preparation.

AuNR-peptide aptamer conjugates were prepared by adding 8 μl of the peptide aptamer (concentration 1.31 mM in water), 2 μl at a time to a solution of 1 ml of twice centrifuged nanorods. The solution was left overnight on a shaker to homogenize the conjugation. The resulting nanorod-peptide aptamer conjugates showed a shift of ˜3 nm.

AuNR-Troponin antibody conjugation preparation.

A solution was prepared by adding 67 μl of heterobifunctional polyethylene glycol (SH-PEG-COOH) in water (2 μM) with the same molar ratio of EDC and NHS, followed by shaking for one hour. The pH of the above reaction was adjusted to 7.4 by adding 10× concentrated PBS, followed by the addition of 100 μl of anti-Troponin antibody (1.34 μM, Mw=150 kDa) (H-41; Santa Cruz Biotechnology, Inc., Dallas, Tex.). The reaction mixture was incubated for an additional two hours and filtered to remove excess chemicals and byproducts by centrifugation using a centrifuge tube with 50 kDa filter. The conjugate mixture was obtained by washing the conjugates with water twice. The AuNR-antibody cojugate mixture was obtained by adding 6 μl of SH-PEG-antibody conjugate of concentration 1.34 μM to 1 ml of twice centrifuged nanorods. The affinity of the SH-PEG-antibody was the same as pristine antibody was confirmed by dot-blot analysis.

Bioplasmonic Paper Substrate (Paper-based Plasmonic Biosensor) Preparation.

A regular laboratory filter paper (Whatman #1) was used for the absorption of nanorods. A 1 cm ×1 cm paper strip was immersed in a solution of AuNR conjugates (peptide aptamer and antibody) and left overnight at 4° C. The paper strip was washed with tris buffer and immersed in different concentrations of troponin in tris buffer (pH 8) for 2 hours at 4° C. After immersion, the paper strips were thoroughly washed with tris buffer and dried under a stream of nitrogen.

Spiked Human Serum and Artificial Sweat Tests.

Troponin of various concentrations was used to spike human serum ( 1/10th concentration) in tris buffer (pH 8). Similarly, Troponin of various concentrations was used to spike artificial sweat ( 1/10th concentration). Bioplasmonic paper as prepared above was immersed in these solutions and left at 4° C. for two hours. It was removed and washed thoroughly with tris buffer to remove all the non-specific binding.

Extinction Spectra Measurements.

Extinction spectra from bioplasmonic paper substrates were collected using a CRAIC microspectrophotometer (QDI 302) coupled to a Leica optical microscope (DM 4000 M) with 20× objective in the range of 450-800 nm with 10 accumulations and 100 ms exposure time in reflection mode. The spectral resolution of the microspectrophotometer is 0.2 nm. Multiple UV-Visible spectra (˜10) were collected from different locations of the bioplasmonic paper strip before and after exposure to troponin solution. Shimadzu UV-1800 spectrophotometer was employed to collect UV-Vis extinction spectra from solution.

Layer-by-Layer Assembly.

Glass substrates were modified by 1% P2VP followed by the adsorption of AuNRs. For layer-by-layer assembly, AuNR substrates were immersed in 1 wt. % PSS in 0.1 M NaCl aqueous solution for 15 minutes followed by rinsing with DI-H₂O water for 30 seconds and rinsing with 0.1 M NaCl solution for an additional 30 seconds on each side of the glass slides. Then the substrates were immersed in a solution of 1 wt. % PAH in 0.1 M NaCl for 15 minutes followed by the rinsing procedure described above. Subsequently, the substrates were dried by nitrogen stream before obtaining extinction spectra with an UV-Vis spectrometer. The above procedure was repeated 10 times to deposit a total of 10 bilayers. The thickness of each polyeletrolyte bilayer was ˜2 nm.

Electron Microscopy Characterization.

Transmission electron microscopy (TEM) micrographs were recorded on a JEM-2100F (JEOL) field emission instrument. Samples were prepared by drying a drop of the solution on a carbon coated grid, which had been previously made hydrophilic by glow discharge. Scanning electron microscope images were obtained using a FEI Nova 2300 Field Emission SEM at an accelerating voltage of 10 kV. The paper was gold sputtered for 60 second before SEM imaging.

### Example 1

Cardiac Troponin I (cTI) Paper-Based Plasmonic Biosensor.

In this Example, human cardiac troponin I peptide was used to prepare human troponin I peptide aptamer-based plasmonic biosensors and investigate their sensitivity and stability as compared to antibody-based plasmonic biosensors.

Unique linear peptide motifs for human Troponin I peptide of nM affinity were identified by polyvalent phage display as described in Park et al. (Biotechnol. Bioeng. 2010, 105(4):678-686) and Wu et al. (Anal. Chem. 2010, 82:8235-8243). Peptide aptamers and antibodies as biological recognition elements (BRE) were coupled to gold nanorods (AuNR) to prepare plasmonic biosensors. Filter paper substrates were uniformly coated with BRE-AuNR.

As demonstrated herein, plasmonic biosensors with peptide biological recognition elements provide a significantly higher sensitivity and lower limit of detection (few pg/ml) compared to natural antibodies as a recognition element. Furthermore, plasmonic biosensors with peptide biological recognition elements exhibited excellent stability by retaining their target-recognition capability at high temperatures unlike plasmonic biosensors with antibodies biological recognition elements which lose their recognition functionality.

### Example 2

In this Example, peptide aptamer-based plasmonic biosensors were analyzed to determine the refractive index sensitivity.

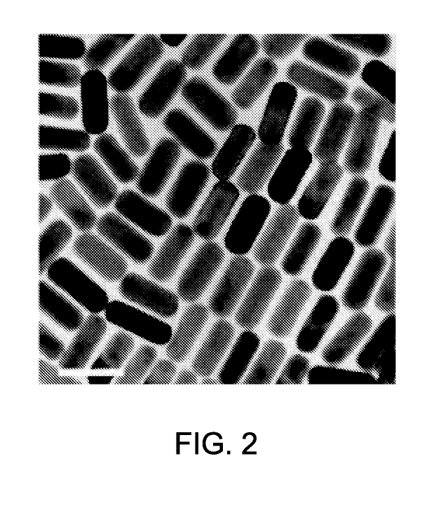

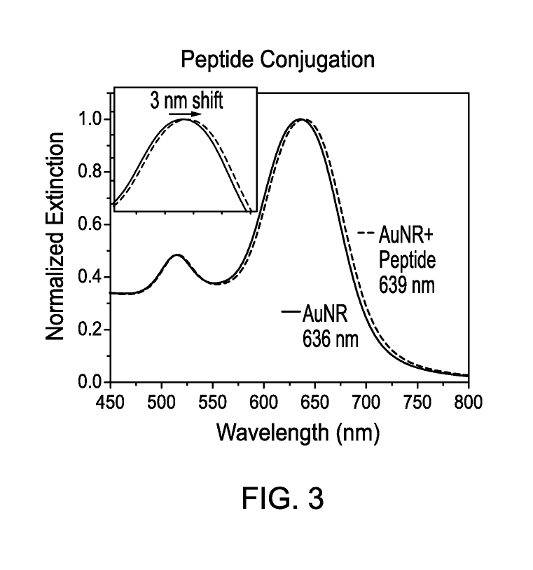

AuNRs were synthesized using a seed-mediated approach with a length of 47.3±2.3 nm and a diameter of 20.2±1.4 nm (FIG. 2). The conjugation of peptides to AuNRs was achieved by adding a cysteine residue at the C-terminus of the peptide aptamer chain to facilitate binding of the peptide aptamers to AuNRs through a Au-S linkage. After the binding of the peptide aptamer to the surface of the nanorod, the LSPR wavelength of the nanorod exhibited a red shift of ˜3 nm due to the increase in the refractive index of the medium surrounding AuNR (FIG. 3).

### Example 3

In this Example, the mechanism for how the peptide aptamer binds to troponin I was investigated.

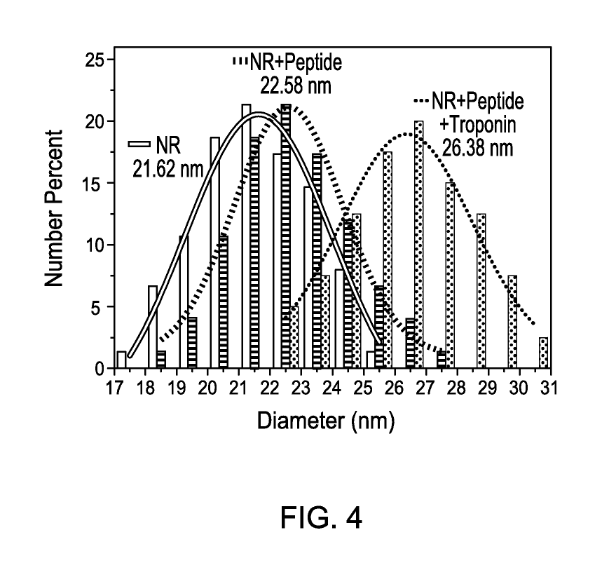

To experimentally probe the troponin binding to the peptide aptamer, peptide aptamer-conjugated AuNRs were exposed to troponin at a concentration of 3.53 μg/ml. After binding, AFM images revealed that the diameter of AuNR increased by ˜3.8 nm (FIG. 4). Adding the thickness of the peptide aptamer in dry state (˜0.9 nm), the thickness of the protein on the surface of the AuNR was about 4.7 nm.

### Example 4

In this Example, paper substrates with peptide aptamer-based plasmonic biosensors were investigated.

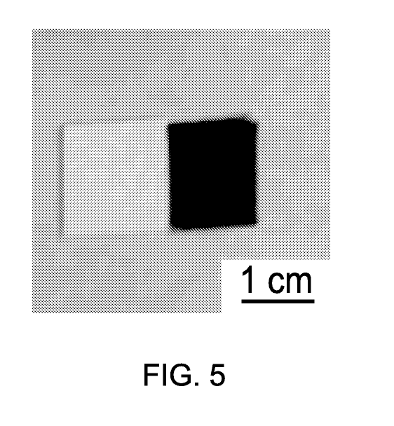

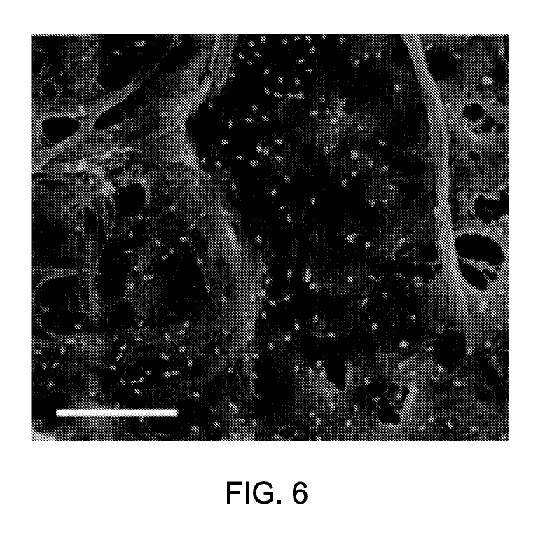

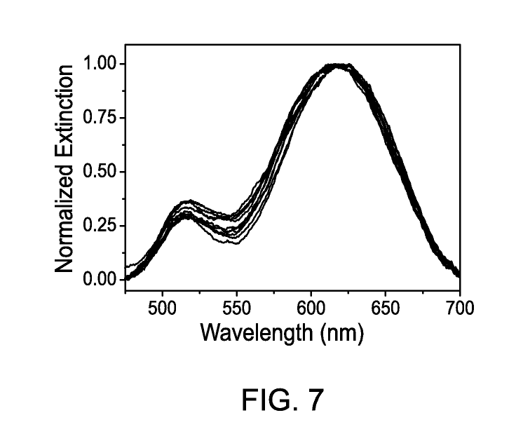

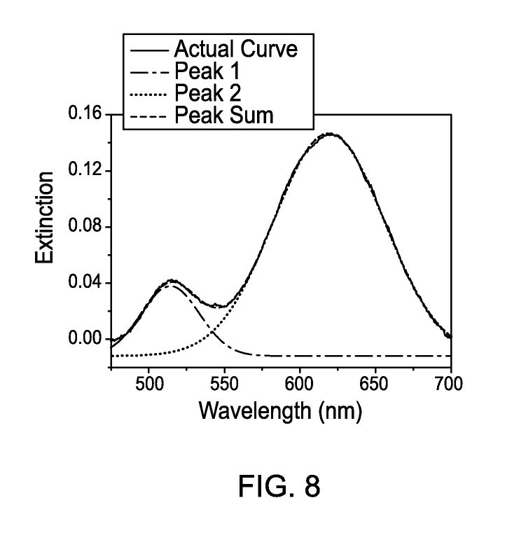

Peptide aptamer-conjugated AuNRs were adsorbed on a filter paper by immersion of a 1×1 cm strip of filter paper in a solution of peptide aptamer-conjugated AuNR. FIG. 5 shows a photograph of a filter paper strip without AuNRs (left paper) and a filter paper strip after immersion in AuNRs (right paper). An SEM image of the paper revealed a uniform distribution of peptide aptamer-conjugated AuNRs with no signs of aggregation or patchiness (FIG. 6). The extinction spectra collected from different points across the surface of the paper (1×1 cm) showed excellent optical uniformity with a standard deviation of less than 1 nm in LSPR wavelength (FIG. 7). Each extinction spectrum was baseline subtracted and deconvoluted using a two peak Gaussian fit (FIG. 8).

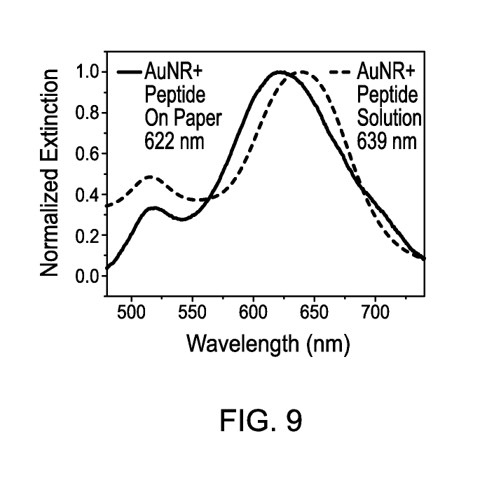

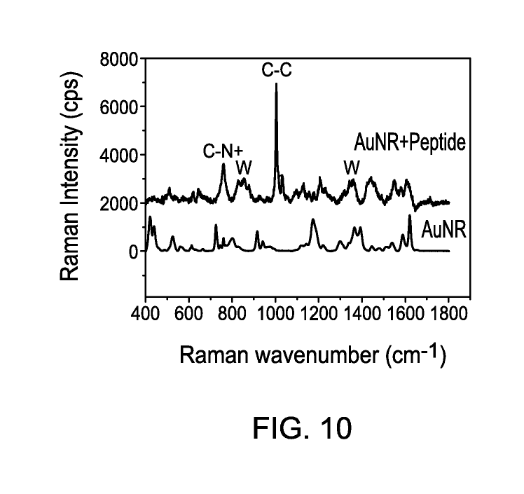

After the adsorption of AuNR on paper, the LSPR wavelength of AuNR exhibited a blue shift of ˜17 nm compared to that in solution due to the decrease in the effective refractive index of the surrounding medium from water to air+paper substrate (FIG. 9). The conjugation of peptide aptamers to the AuNR surface was confirmed using surface enhanced Raman scattering (SERS) spectra obtained from the paper substrates. SERS spectra obtained from the paper substrates revealed Raman bands corresponding to C—C, C—N⁺ vibrations at 1004 cm⁻¹ from phenylalanine and 1341 cm⁻¹, 1360 cm⁻¹ from tryptophan (FIG. 10).

### Example 5

In this Example, the efficacy of the plasmonic biosensor based on peptide aptamers as biological recognition elements was investigated.

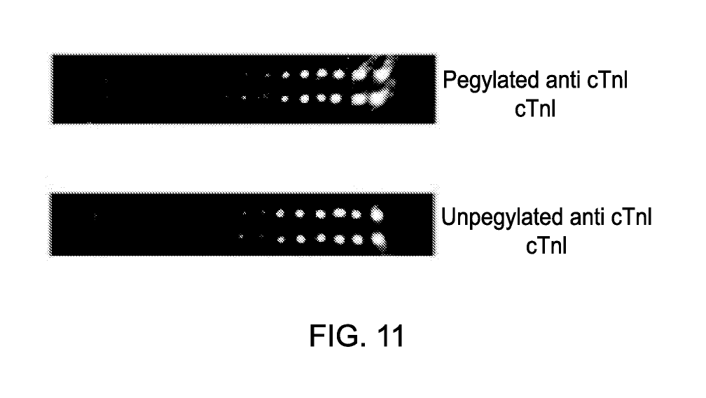

The performance of plasmonic biosensors based on peptide aptamers as recognition elements were compared to natural antibodies as recognition elements. An anti-cTnI polyclonal antibody (H-41; Santa Cruz Biotechnology, Inc., Dallas, Tex.) was used as the conventional recognition element. The antibody was conjugated to the AuNR using carbodiimide crosslinker chemistry and thiol-terminated poly(ethylene glycol) (SH-PEG) (as discussed in the Experimental Section). Dot-blot was used to confirm that the affinity of the antibody towards troponin was preserved after bioconjugation with SH-PEG as shown in FIG. 11. The thiol terminus bound to the surface of the nanotransducer via an Au—S linkage.

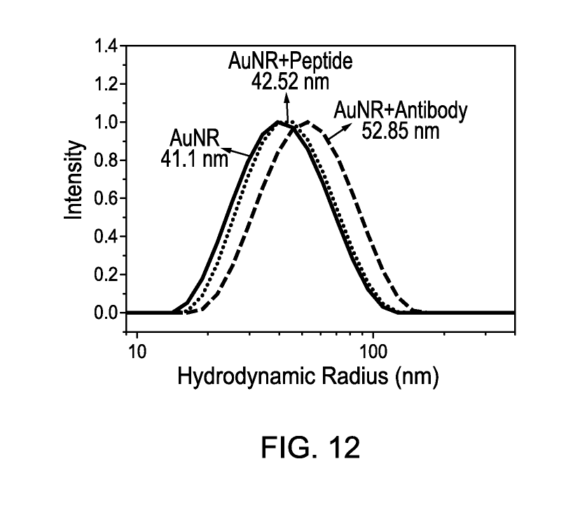

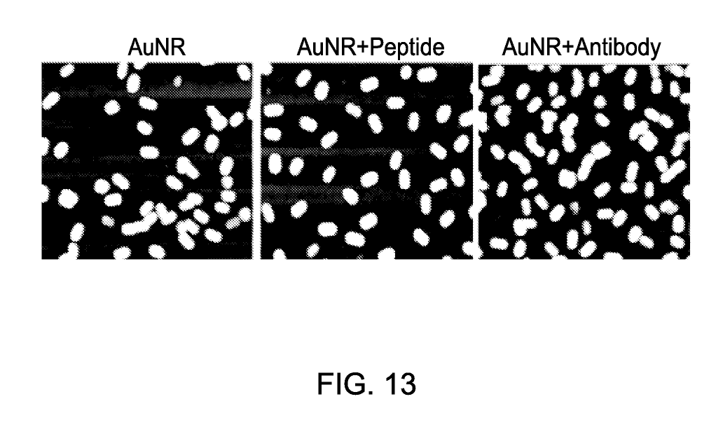

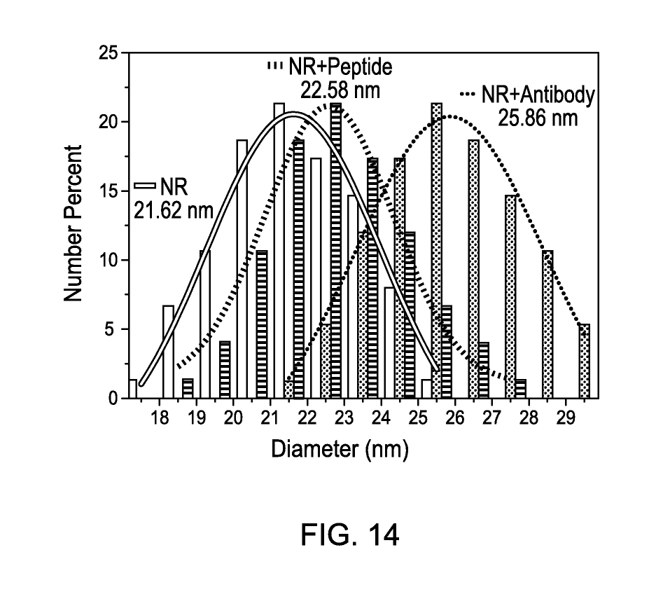

Conjugation of the antibody to the AuNR resulted in a red shift of in the LSPR wavelength. Dynamic light scattering (DLS) was used to monitor the changes in the hydrodynamic size of the nanostructures upon bioconjugation of AuNR with peptide aptamers and antibodies. The increase in the hydrodynamic radius of peptide aptamer-conjugated AuNR (˜1.4 nm) was much smaller than antibody-conjugated AuNR (˜11.8 nm) due to the significantly smaller size of peptide aptamer (1640 Da) compared to the antibody (150 kDa) (FIG. 12). The dry state thickness of the peptide aptamer and antibody recognition layers was measured using AFM. AFM images of AuNR, AuNR+peptide aptamer and AuNR+antibody are show in FIG. 13. These results demonstrated that the increase in the diameter of AuNRs after antibody conjugation (˜4.2 nm) was significantly higher than peptide aptamer conjugation (˜1 nm) (FIG. 14).

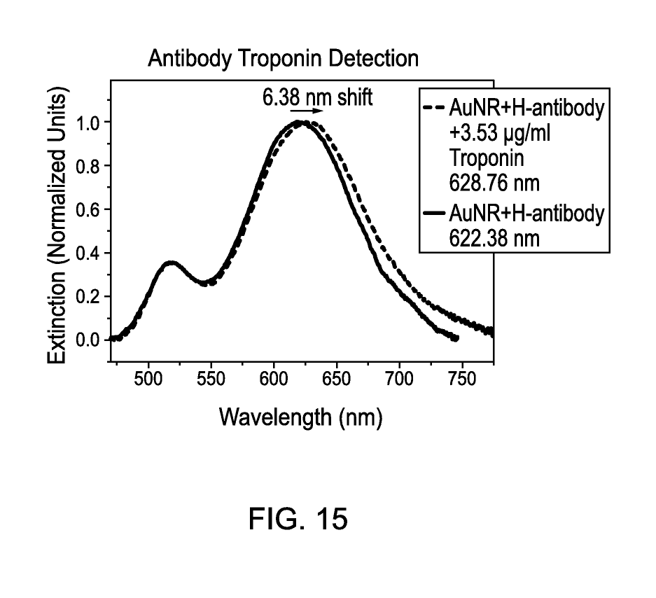

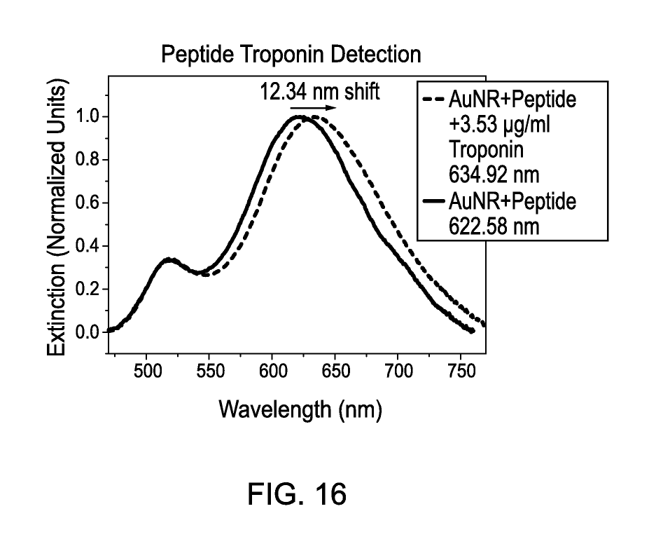

The areal density of the AuNR-antibody and AuNR-peptide aptamer biosensors adsorbed on the paper surface was found to be 52±3/μm² and 49±2/μm². The density of the AuNRs was similar in both cases thus devoiding any effects on the sensitivity due to variations in density. The extinction spectrum from the paper was collected from a 2×2 μm² area, which corresponded to ˜200 nanorods determined using a microspectrometer mounted on an optical microscope. To probe the biosensing capability of the bioplasmonic paper substrates having AuNR-antibody biosensors and AuNR-peptide aptamer biosensors, the paper substrates were exposed to troponin (3.53 μg/ml) in tris buffer. The extinction spectra obtained from antibody-AuNR on paper after exposure to 3.53 μg/ml cTnI showed a red shift of 6.3 nm (FIG. 15) whereas the extinction spectra obtained from peptide aptamer-AuNR on paper after exposure to 3.53 μg/ml cTnI showed a red shift of 12.3 nm (FIG. 16). The difference in the shifts can be attributed to difference in sensing distance from the nanotransducer.

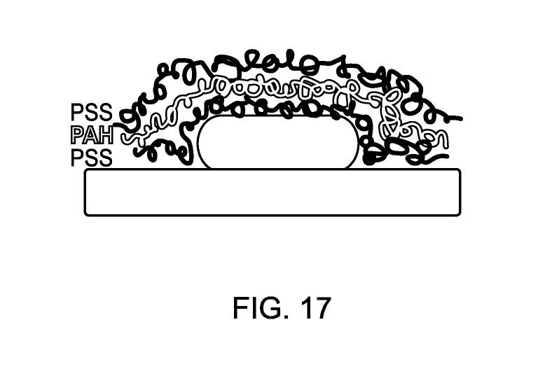

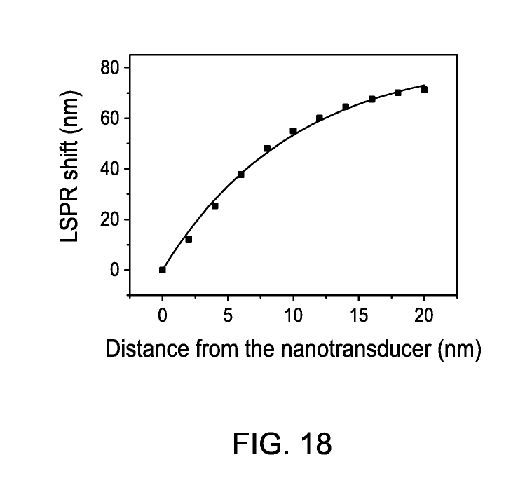

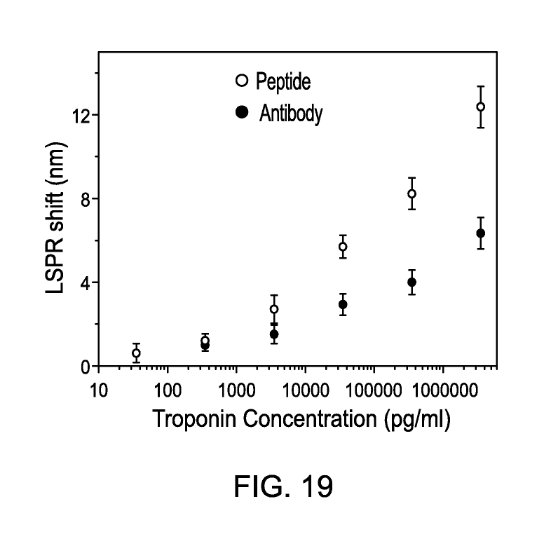

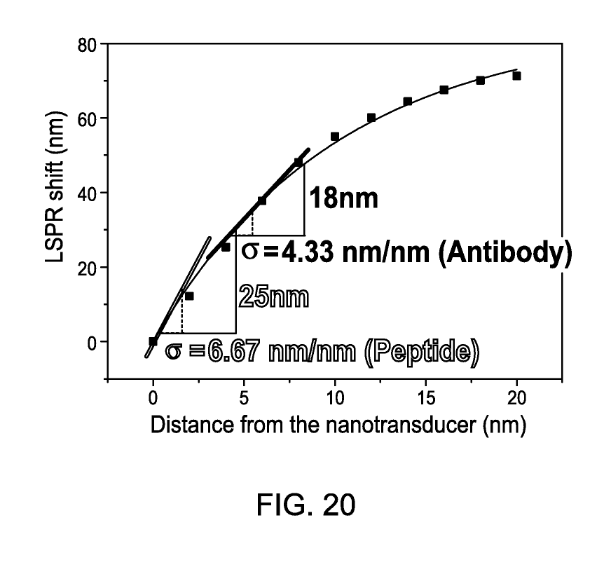

To understand the distance dependent refractive index sensitivity, polyelectrolyte multilayers were deposited on AuNR using a layer-by-layer assembly method to determine the refractive index sensitivity and EM decay length (see schematic in FIG. 17). Extinction spectrum was collected after the deposition of each bilayer and the spectrum was deconvoluted to obtain the longitudinal LSPR wavelength. The red shift in LSPR wavelength with the increase in the thickness of the polyelectrolyte multilayers exhibited a near exponential behavior (FIG. 18). The troponin sensing calibration curve for both peptide aptamer conjugated AuNR and antibody conjugated AuNR is shown in FIG. 19). The distance-dependent refractive index sensitivity (σ), which is the LSPR shift caused by the deposition of 1 nm thick dielectric layer (polyelectrolyte multilayers in the present case) at predetermined distance from the surface of the nanotransducer was deduced at different distances from the AuNR surface from the plot shown in FIG. 18. The extinction measurements after troponin binding to the bioconjugated AuNR were performed in dry state. Considering that the thickness of peptide aptamer and antibody in dry state was 1 nm and 4.2 nm, respectively, the values of σ were computed as shown in FIG. 20. For peptide aptamer recognition elements, σ was determined to be 6.67 nm/nm whereas the antibody recognition element σ was determined to be 4.33 nm/nm. The ratio of σₚₑₚ to σ_{antibody} was 1.54.

The value of σ obtained from the distance dependence curve was compared to the slope of the curves obtained from troponin calibration curve. The calibration curves obtained for troponin sensing in the linear regime fit to a linear curve (FIG. 20). The ratio of the slopes from the calibration curve for peptide was 2.07. The difference between this value and σₚₑₚ:σ_{antibody} was attributed to the peptide aptamer and antibody not being in a completely dry state during the measurements and the possible compression of some of the organic film on the surface of the nanostructure by the AFM tapping mode. The σₚₑₚ:σ_{antibody} could not be compared to absolute ratio of e-d1 from equation (2) because the model assumes a 1:1 binding of multivariant analytes with an invariant binding to the surface capped ligand. This is not true in case of chemically synthesized AuNR, which have different CTAB coating across the surface. The affinity of troponin will not be homogenous across the surface and the refractive index change associated with different points of binding will be different. The limit of detection in case peptide conjugated AuNR was 35.3 μg/ml, which is an order of magnitude lower than that of antibody conjugated AuNR 353 μg/ml. But the sensitivity of the peptide is twice that of the antibody.

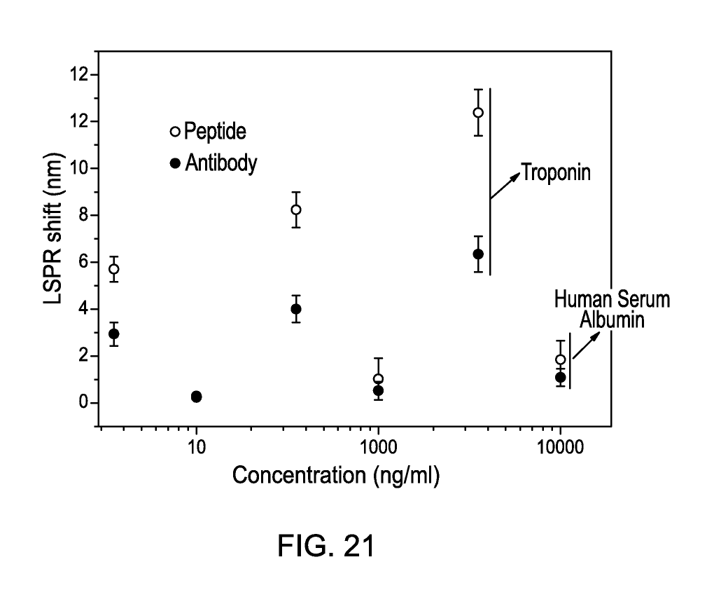

To compare the selectivity of the peptide and antibody towards troponin, human serum albumin (HSA) was used as an interfering protein (FIG. 21). The non-specific binding of the interfering molecule in case of antibody was lower than that of the peptide indicating that the affinity of Troponin towards the antibody was higher than that of the peptide. Although the affinity of the antibody towards troponin was higher, the sensitivity of the peptide was remarkable when compared to antibody because of distance dependent analyte sensitivity (σ) value.

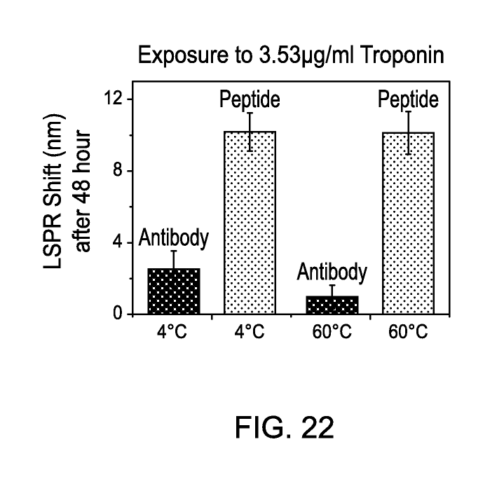

In addition to the differences in sensitivities, peptides exhibit temperature stability unlike protein antibodies that denature at higher temperature. To investigate this for peptide-based biosensors and antibody-based biosensors, the biosensors were exposed to temperatures as high at 60° C. for a period of 48 hours (FIG. 22). When the peptide-based biosensors were exposed to Troponin at a concentration of 3.53 μg/ml, the peptide-based biosensor exhibited remarkable stability shown as a consistent shift in the LSPR. On the other hand, the antibody-based biosensor showed a lower shift even at 4° C. for 48 hours. The remarkable temperature stability of the peptide-based biosensors allows for use of the peptide-based biosensors in point-of-care applications.

### Example 6

In this Example, peptide-based biosensors were used to detect troponin in physiological fluids using peptide-based biosensors.

Troponin in human serum is an indicator of myocardial infarction. Peptide conjugated AuNR were used to sense troponin spiked in human serum ( 1/10th concentration). To reduce the effects of non-specific binding, the paper substrates were exposed to 1% Human Serum Albumin to block the non-specific binding sites.

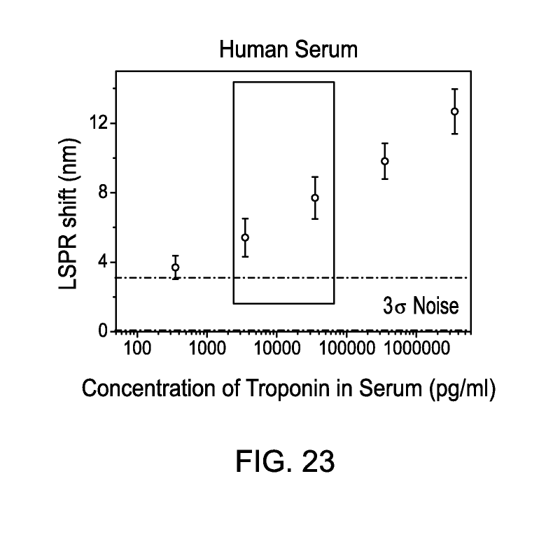

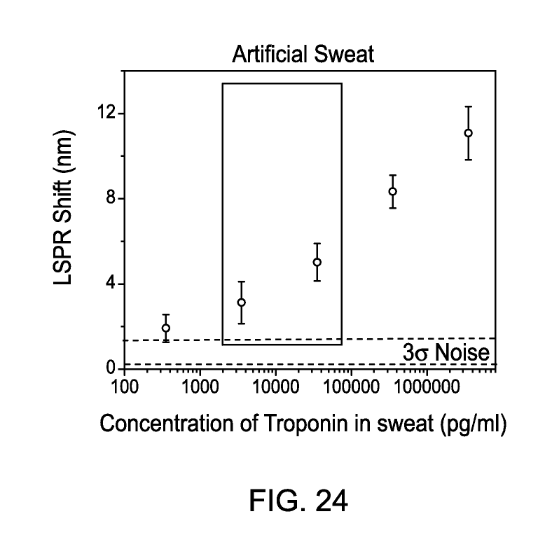

The sensing calibration curve is shown in FIG. 23. The physiologically relevant concentration is 100 pg/ml-1 ng/ml. The LSPR shift was greater than the noise level 3σ to demonstrate the utility of this technique. In addition, Troponin was sensed in artificial eccrine in physiological relevant concentrations as shown in FIG. 24.

The use of antibody-based plasmonic biosensors results in a lower sensitivity due to a relatively higher distance of the epitope from the surface of the plasmonic nanostructure in comparison to peptide-based plasmonic biosensors according to equation (2). Plasmonic nanotransducers have distance dependent refractive index sensitivity, which enables peptide-based plasmonic biosensors to have a greater sensitivity when compared to antibody-based plasmonic biosensors because of their compact structure.

These Examples demonstrate the preparation and use of plasmonic nanotransducers having peptide aptamers and nanostructure cores. The plasmonic nanotransducers provide a label-free method for detecting any target molecule of interest in biological samples. Peptide aptamer recognition elements provide a significantly higher sensitivity and lower limit of detection (few pg/ml) compared to natural antibodies as a recognition element. Furthermore, peptide aptamers exhibit excellent stability by retaining their target-recognition capability at high temperatures unlike antibodies which lose their recognition functionality.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the scope and concept of the disclosure as defined by the appended claims.

What is claimed is:

1. A plasmonic nanotransducer comprising a nanostructure core and a peptide aptamer coupled to the hollow nanostructure core, wherein the peptide aptamer specifically binds to a biomarker.

2. The plasmonic nanotransducer of claim 1 wherein the nanostructure core comprises a hollow nanostructure core.

3. The plasmonic nanotransducer of claim 2 wherein the hollow nanostructure core is selected from the group consisting of a nanocage, a nanorattle, a nanoshell, and a nanomatryoshka.

4. The plasmonic nanotransducer of claim 1 adsorbed to a substrate.

5. The plasmonic nanotransducer of claim 4 wherein the substrate is selected from the group consisting of a glass substrate, a paper substrate, and a fibrous mat.

6. The plasmonic nanotransducer of claim 5 wherein the glass substrate is selected from the group consisting of silica, titania, and alumina.

7. The plasmonic nanotransducer of claim 5 wherein the paper substrate is selected from the group consisting of cellulose paper, nitrocellulose paper, methylcellulose paper, hydroxypropylcellulose paper, and nanocellulose paper.

8. The plasmonic nanotransducer of claim 5 wherein the fibrous mat is selected from the group consisting of a woven fibrous mat and a non-woven fibrous mat.

9. A label-free method for detecting a biomarker in a biological sample, the method comprising:
obtaining a biological sample from the subject;
contacting the biological sample with a plasmonic nanotransducer, wherein the plasmonic nanotransducer comprises:
a nanostructure core; and
at least one peptide aptamer coupled to the nanostructure core, wherein the at least one peptide aptamer specifically binds to a biomarker;
wherein the biomarker in the biological sample forms a complex with the plasmonic nanotransducer; and
detecting the complex.

10. The method of claim 9 wherein the complex is detected using a method selected from the group consisting of local surface plasmon resonance and surface enhanced Raman scattering.

11. The method of claim 9 wherein the nanostructure core comprises a hollow nanostructure core.

12. The method of claim 11 wherein the hollow nanostructure core is selected from the group consisting of a nanocage, a nanorattle, a nanoshell, and a nanomatryoshka.

13. The method of claim 9 wherein the nanostructure core is selected from the group consisting of a gold nanostructure core, a silver nanostructure core, a copper nanostructure core, and combinations thereof.

14. The method of claim 9 wherein the biological sample comprises a liquid biological sample.

15. The method of claim 14 wherein the liquid biological sample is selected from the group consisting of whole blood, plasma, serum, urine, saliva, cerebrospinal fluid, and sweat.

16. The method of claim 15 wherein the target molecule is selected from the group consisting of a cell, a protein, a peptide, a nucleic acid, and combinations thereof.

17. The method of claim 9 wherein the biomarker is selected from the group consisting of a cardiac biomarker, a cancer biomarker, a kidney disease biomarker, an aging biomarker, a hospital-acquired infection biomarker, and a food poisoning biomarker.

18. The method of claim 17 wherein the cardiac biomarker is selected from the group consisting of troponins such as troponin I (cTI), fatty acid-binding protein 3 (FABP3) creatine kinase-MB, lactate dehydrogenase, aspartate transaminase, myoglobin, ischemia-modified albumin, B-type natriuretic peptide (BNP), N-terminal fragment of pro-BNP (NT-proBNP), Mid-regional pro-Atrial Natriuretic Peptide, glycogen phosphorylase isoenzyme BB, soluble urokinase-type plasminogen activator receptor, copeptin, myeloperoxidase (MPO), growth differentiation factor 15 (GDF-15), high sensitivity C-reactive protein (hsCRP), placental growth factor (P1GF), whole blood choline (WBCHO), interleukin 1 receptor-like 1 (ST2), C-Terminal pro-endothelin 1, Mid-regional pro-Adrenomedullin, and combinations thereof.

19. The method of claim 17 wherein the kidney disease biomarker is selected from the group consisting of serum creatinine (SCr), cystatin C (CyC), neutrophil gelatinase-associated lipocalin (NGAL), kidney injury molecule-1 (KIM-1), β-Trace protein (BTP), uric acid (UA), proteinuria, albumin, liver-fatty acid binding protein (L-FABP), interleukin-18 (IL-18), urine cystatin C (uCyC), Alpha-glutathione s-transferase (α-GST), pi-glutathione s-transferase (π-GST), gammaglutanyl transpeptidase (GGT), alkaline phosphatase (AP), N-acetyl-β-D-glucosaminidase (NAG), tenascin, tissue inhibitor of metalloproteinases 1, nephrin, podocin, podocalyxin, asymmetric dimethylarginine (ADMA), C-reactive protein (CRP), soluble tumor nectosis factor receptor II, pentraxin-3 (PTX3), transforming growth factor-β1 (TGF-β1), CD14, fibroblast growth factor-23 (FGF-23), apolipoprotein A-IV, adiponectin, γ-glutamyl transpeptidase (GGT), Tumor necrosis factor-related apoptosis-inducing ligand (TRAIL), bone morphogenetic protein-7 (BMP-7), and combinations thereof.

20. The method of claim 17, wherein the cancer biomarker is selected from the group consisting of alpha-fetoprotein (AFP), breakpoint cluster region-Abelson murine leukemia viral oncogene homolog 1 (BCR-ABL), breast cancer 1 (BRCA1), breast cancer 2 (BRCA2), V-Raf Murine Sarcoma Viral Oncogene Homolog B1 (BRAF V600E), cancer antigen-125 (CA-125), carbohydrate antigen 19-9 (CA19.9), carcinoembryonic antigen (CEA), epidermal growth factor receptor (EGFR), human epidermal growth factor receptor 2 (HER-2), Mast/stem cell growth factor receptor (KIT, CD117), prostate-specific antigen (PSA), S100, fatty acid-binding protein 3 (FABP3), aquaporin-1 (AQP1), perilipin 2 (PLIN2), and combinations thereof.
